(19) United States
(12) Patent Application Publication
(10) Pub. No.: US 2006/0161156 A1
(43) Pub. Date: Jul. 20, 2006

(54) Fracture fixation device

(76) Inventor: Jorge L. Orbay, Miami, FL (US)

Correspondence Address: GORDON & JACOBSON, P.C., 60 LONG RIDGE ROAD, SUITE 407, STAMFORD, CT, 06902, US

(21) Appl. No.: 11/384,842

(22) Filed: Mar. 20, 2006

Related U.S. Application Data
(63) Continuation-in-part of application No. 10/515,699, filed on Nov. 25, 2005.
(63) Continuation-in-part of application No. 11/040,732, filed on Jan. 21, 2005.
(60) Provisional application No. 60/643,432, filed on Jan. 7, 2005.
(60) Provisional application No. 60/546,127, filed on Feb. 20, 2004.

(51) Int. Cl. A61F 2/30

(52) U.S. Cl. 606/62

(57) ABSTRACT

A fracture fixation device includes a plate portion and an intramedullary nail portion which is offset relative to the plate portion by a neck portion. The plate portion overhangs the neck portion and includes longitudinally displaced holes which orient pegs along an imaginary surface parallel to subchondral bone of an articular surface. The nail portion includes threaded screw holes oriented normal to an endosteal surface, and a smaller K-wire alignment hole parallel to the screw holes. The jig has a first portion which references with the dimple and a second portion in alignment over the screw holes of the nail portion. The back of the first portion of the jig is curved upward to facilitate maneuvering of the jig. The first and second portions of the jig includes K-wire guide holes which direct K-wires relative to holes in the device.

### RELATED APPLICATIONS

This application is a continuation-in-part of U.S. Ser. No. 10/515,699, filed Nov. 25, 2005, which is a national stage application of PCT/US03/14775, filed May 69, 2003, which claims priority from U.S. Ser. No. 10/315,787, now issued as U.S. Pat. No. No. 6,706,046, which is a continuation-in-part of U.S. Ser. No. 10/159,611, now issued as U.S. Pat. No. 6,730,090; this application is also a continuation-in-part of U.S. Ser. No. 11/040,732, filed Jan. 21, 2005, which claims the benefit of U.S. Provisional App. Nos. 60/643,432, filed Jan. 7, 2005, and 60/546,127, filed Feb. 20, 2004; and this application claims the benefit of U.S. Provisional App. No. 60/648,989, filed Jan. 28, 2005, all of the above being hereby incorporated by reference herein in their entireties. The application is also related to U.S. Pat. No. 6,926,720, which is hereby incorporated by reference herein in its entirety.

### BACKGROUND OF THE INVENTION

1. Field of the Invention

This invention relates broadly to surgical devices. More particularly, this invention relates to surgical devices and tools for implanting fracture fixation devices.

2. State of the Art

Severe long bone fractures are often treated with plating. In plating, a relatively large incision is made at the location of the fracture, musculature and tendons are displaced from the bone to expose the bone surface, and a bone plate is fixedly attached to one or more pieces of the fractured bone in a manner which, ideally, supports and stabilizes the fracture for healing. Due to the relatively invasive nature of the procedure required to implant the plate, plating is generally reserved for fractures which cannot be treated with a less invasive method of immobilization.

Less complicated fractures are often treated with casting or wires. However, such conservative treatment may not provide the stabilization and support necessary for desirable recovery. Yet, the operative procedure of plating is often too invasive for the relative non-severity of the fracture. Moreover, conventional plating can result in tendon irritation and skin necrosis, and may require extensive periosteal stripping in order to apply the plate on the bone surface. As such, many of the less displaced fractures, and particularly metaphyseal fractures (fractures at the end of the long bones), remain under-treated.

By way of example, a Colles' fracture, which results from compressive forces being placed on the distal radius bone, and which causes backward displacement of the distal fragment and radial deviation of the hand at the wrist, is treated with a dorsal plate when there is a significant degree of displacement. However, a less-displaced Colles' fracture is commonly under-treated due to the hesitancy of physicians to prescribe operative and invasive treatment. If not properly treated, such a fracture results in permanent wrist deformity. It is therefore important to align the fracture and fixate the bones relative to each other so that proper healing may occur.

More recently, relatively minimally invasive fixation devices have become available for treatment of wrist fractures. For example. U.S. Pat. No. 6,379,359 to Dahners teaches an intrafocal fixation device including an intramedullary portion and a plate portion which seats at the metaphysis. However, devices like that described in Dahners are not likely to obtain acceptance by surgeons or be effective without addressing several critical issues. First, the subchondral bone must be properly supported. Second, the support must be properly aligned on the bone. Third, the support for the subchondral bone needs to be applied in a manner which is not counter to the minimally invasive design of the implant. Fourth, the implant should not necessitate undue removal of bone, which may further weaken the fracture location. It is therefore necessary to provide to the surgeon with a fracture fixation implant and a jig facilitating the implant thereof.

### SUMMARY OF THE INVENTION

It is therefore an object of the invention to provide a fixation device and a jig for assistance in implantation of the fixation device relative to a fractured bone.

It is another object of the invention to provide a fixation device designed to treat metaphyseal fractures.

It is a further object of the invention to provide a jig assembly which provides proper alignment between longitudinally displaced holes drilled in bone and corresponding openings in an intramedullary portion of the fixation device such that fasteners can be inserted through the holes and openings.

It is an additional object of the invention to provide a jig assembly which facilitates drilling of axially aligned holes through peg holes in a plate of a fixation device and through metaphyseal bone.

In accord with these objects, which will be discussed in detail below, a fracture fixation device and a jig therefor are provided.

A fracture fixation device according to the invention a supra-metaphyseal plate portion and an intramedullary nail portion which is horizontally and vertically offset relative to the plate portion by a neck portion. The plate portion overhangs the neck portion. The plate portion includes longitudinally displaced fixed angle holes, each of which is adapted to orient a peg (or locking screw, collectively referred to as ‘peg’) in a different angular orientation such that pegs therethrough generally corresponds to the articular surface of the subchondral bone. The nail portion includes threaded screw holes oriented normal to an endosteal surface, and a smaller K-wire alignment hole parallel to the screw holes.

An implantation jig for the fixation device has a first portion with a concave surface seatable on the plate portion of the fixation device, and a relatively elevated second portion in alignment over the screw holes of the intramedullary nail portion of the fixation device.

The first portion includes openings in alignment with the fixed angle holes. A cannulated locking drill guide locks the jig relative to the implant and is used to guide a drill in alignment with one of the fixed angle holes. The back of the first portion of the jig is curved upward to facilitate maneuvering the nail portion of the fixation device within the intrafocal space and to allow the first portion to rest on the diaphyseal-side of the fracture during the implantation process. The first portion of the jig includes two K-wire guide holes which extend on either side of the front end of plate portion when the jig is coupled to the plate portion. The K-wire guide holes are designed to closely hold an appropriately sized K-wire and direct it at a fixed angle parallel to the endmost screw hole of the plate portion. Two such guide holes are provided, as the endmost holes for left and right fixation devices (for left and right hands) each have opposite respective angles, and the jig may be used with each of the left and right devices. For a particular application, the K-wire guide hole located on the side of the operative limb (left or right hand) is used. The respective K-wire guide hole defines an axis which is substantially parallel to and closely spaced to the axis of the endmost screw hole, thus anticipating the path of a peg through the endmost screw hole. In use, prior to drilling any holes into bone for the pegs for the screw holes, a K-wire is drilled into bone through the appropriate K-wire guide hole and its placement within the bone is viewed under fluoroscopy to ensure its placement, and the subsequent placement of the peg through the endmost screw hole, relative to the articular surface of the subchondral bone is appropriate.

The second portion of the jig includes longitudinally displaced holes or slots for drill guides. The second portion also includes a K-wire guide hole configured to direct a K-wire at a fixed angle into the K-wire alignment hole of the nail portion of the fixation device. Similar to above, prior to drilling any holes into the diaphysis for the screws for the nail portion, a K-wire is drilled through the K-wire guide hole in the second portion and its placement can be viewed under fluoroscopy to ensure its placement relative to the diaphysis is appropriate.

Additional objects and advantages of the invention will become apparent to those skilled in the art upon reference to the detailed description taken in conjunction with the provided figures.

### BRIEF DESCRIPTION OF THE DRAWINGS

FIG. 1 is a side elevation of a fixation device according to the invention;

FIG. 2 is a plan view of a fixation device according to the invention;

FIG. 3 is a bottom view of the plate-portion end of the fixation device of the invention;

FIG. 4 is a plate-portion end view of the fixation device of the invention; and

FIG. 5 is a perspective view of an implantation jig according to the invention coupled to the fixation device of the invention.

### DETAILED DESCRIPTION OF THE PREFERRED EMBODIMENTS

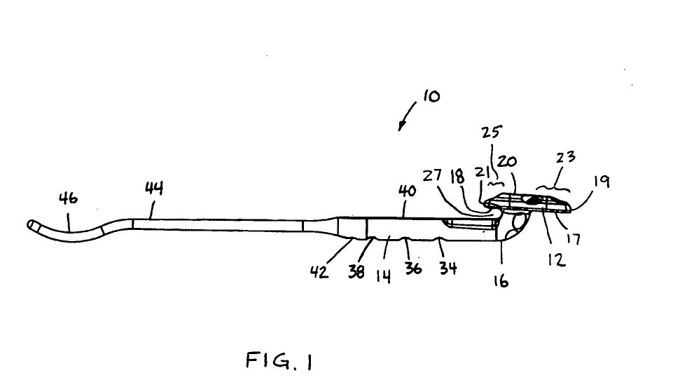

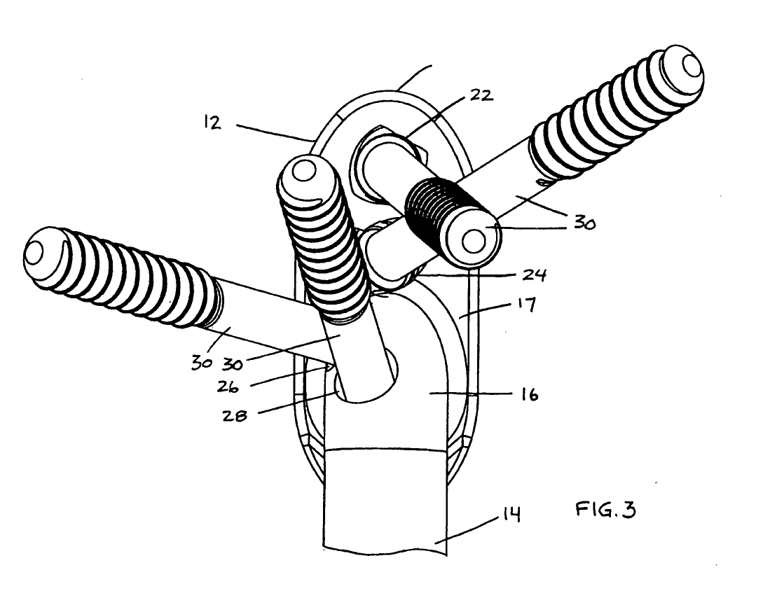

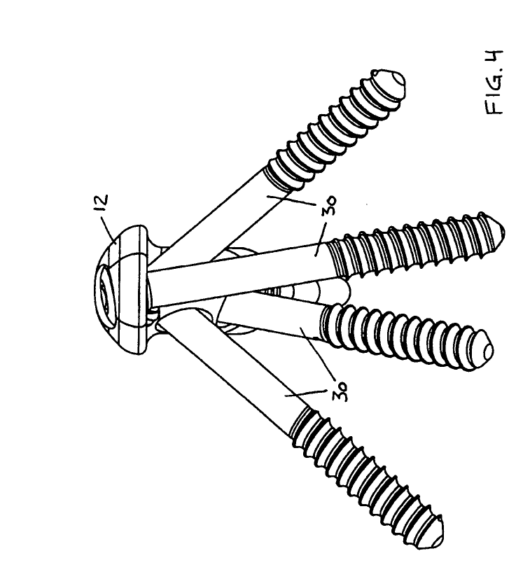

Turning now to FIGS. 1 through 4, a fracture fixation device 10 according to the invention includes a supra-metaphyseal plate portion 12 and an intramedullary nail portion 14 which is horizontally and vertically offset relative to the plate portion. The nail portion extends into a neck portion 16 upon which a lower surface 17 of the plate portion is located, which the neck portion meeting the plate portion inward from first and second ends 19, 21 of the plate portion. The width of the neck portion 16 does not exceed the width of the plate portion 12.

The plate portion has a larger first portion 23 between the neck portion 16 and front end 19 which extends substantially opposite said nail portion 14, and a second portion 25 extending from the neck portion toward the rear end 21 and which defines a lip 18 extending to overhang a portion of said nail portion and a space 27 between the lip and nail portion. This position of the plate portion on the neck portion positions the neck portions for better placement through the fracture line and reduced bone removal for receiving the implant. In addition, the space 27 keys over the edge of the bone increasing implant stability, particularly prior to inserting screws into the nail portion, as discussed below.

The plate portion 12 has a narrow profile and is slightly rounded about its upper surface 20, with the front and rear ends 19, 21 being tapered. The plate portion 12 includes four longitudinally displaced fixed angle holes 22, 24, 26, 28, each preferably threaded, and each of which is adapted to lock a fastener, such as a smooth or threaded shaft peg 30, in a different orientation from the others (i.e., the axes are oblique relative to each other). When viewed from the front end 19 of the device, the pegs 30 are laterally displaced defining an imaginary surface (generally transverse to the longitudinal axis A of the plate portion and a plane in which the nail portion extends) which generally corresponds to (i.e., extends parallel to) the articular surface of the subchondral bone, e.g., at the distal radius. Preferably, exactly two of the fixed angle holes 22, 24 (and associated pegs) extend solely through first portion 23 of the plate portion 12, while two others extend through the second portion 25 and the plate and neck portions 12, 16. The plate portion 12 includes a dimple 32 for referencing an implantation jig, as described below.

The nail portion 14 includes three threaded screw holes 34, 36, 38 oriented normal to a preferably flattened endosteal surface 40, and a smaller K-wire alignment hole 42 parallel to the screw holes and sized to closely receive a K-wire at a fixed angle. The tail end 44 of the nail portion 14 is provided with a portion 46 having downward and then upward curve which facilitates maneuvering the tail end of the nail portion through the intrafocal space and into the medullary canal of the diaphysis, e.g., of the distal radius.

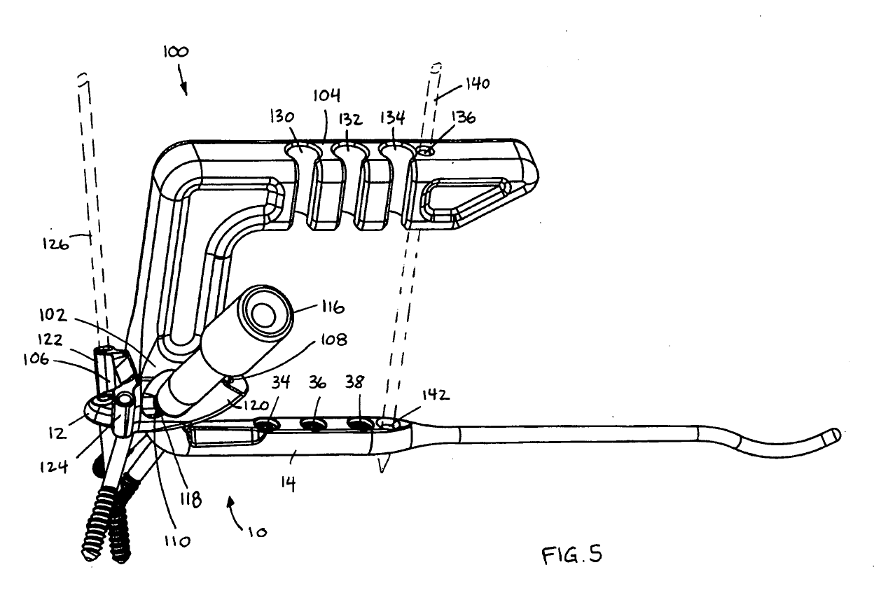

With the fixation device 10 in mind, turning now to FIG. 5, an implantation jig 100 for the fixation device has a first portion 102 seatable on the plate portion 12 of the fixation device, and a relatively elevated second portion 104 in alignment over the screw holes 34, 36, 38 of the intramedullary nail portion 14 of the fixation device 10.

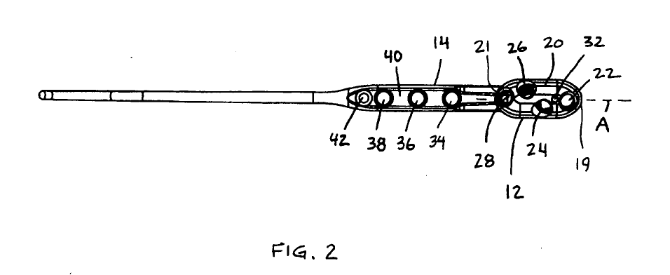

The first portion 102 includes a lower nub (not shown) which seats in the dimple 32 (FIG. 2) on the plate portion 12 of the fixation device. In addition, the first portion includes access openings 106 (at the front), 10 (at the left side, and at right the side, not shown), 108 (at the rear) in axial alignment with the fixed angle screw holes 22, 24, 26, 28. Such openings are preferably initially positioned over drill guide tips (not shown) which are inserted in all of the fixed angle holes of the plate portion but the hole beneath access opening 110. The drill guide tips which functions as miniature drill guides, are described in detail in co-owned U.S. Ser. No. 11/011,917, filed Dec. 14, 2004, which is hereby incorporated by reference herein in its entirety. As another option, the drill guides tips are not necessitated, and individual drill guides can be inserted into the fixed angle holes just prior to drilling holes into bone in alignment with the holes.

In accord with one aspect of the invention, a cannulated locking drill guide 116 having a threaded end 118 is provided through opening 110 and thread into hole 26 in the plate portion 12, such that a portion of the guide 116 applies a force against said jig 100. The locking drill guide 116 in conjunction with the engagement of the nub within the dimple 32 immobilizes the jig 100 relative to the fixation device 10 so that the two are fixed relative to each other.

In accord with another aspect of the invention, the back 120 of the first portion 102 of the jig 100 is curved upward to facilitate maneuvering the nail portion 14 of the fixation device within the intrafocal space and to allow the first portion to rest on the diaphyseal-side of the fracture (e.g., the proximal cortex of the distal radius) during the implantation process.

The plate portion 12 of the fixation device 10 is too narrow to support the inclusion of K-wire guide holes. Thus, in accord with a further aspect of the invention, the first portion 102 of the jig 100 includes two K-wire guide holes 122, 124 which extend on either side of the front of the plate portion when the jig 100 is fixed relative to the fixation deice 10 to guide a K-wire offset relative to the plate portion. The K-wire guide holes 122, 124 are designed to closely hold an appropriately sized K-wire 126 and direct it at a fixed angle parallel to the endmost screw hole 22 (e.g., distalmost where the fixation device is used at the distal radius). Two such guide holes are provided, as the endmost holes for left and right fixation devices (for left and right hands) each have opposite respective angles, and the same jig may be used with each of the left and right devices. For a particular application, the K-wire guide hole located opposite the side on which the locking drill guide is positioned is used. That is, as shown in FIG. 5, with the locking drill guide on the left side of the plate, the right side K-wire guide hole 122 is used. The respective K-wire guide hole 122 defines an axis which is laterally offset, substantially parallel to and closely spaced to the axis of the endmost screw hole 22 (preferably within approximately 5 mm), thus anticipating the path of a peg through the endmost screw hole 22. In use, prior to drilling any holes into bone for the pegs 30 for the screw holes, a K-wire 126 is drilled into bone through the appropriate K-wire guide hole 122 and its placement within the bone is viewed under fluoroscopy to ensure its placement relative to the articular surface of the subchondral bone is appropriate. If correct, the screw holes can be drilled with assurance that the screws will not enter the articular space. If not correctly located as indicated by K-wire fluoroscopic visualization, the plate can be repositioned, the K-wire re-drilled and re-examined until placement is correct.

The second portion 104 of the jig includes longitudinally displaced holes or slots 130, 132, 134, as described in detail in U.S. Pat. No. 6,926,720, to longitudinally align drill guides with the screw holes 34, 36, 38 of the intramedullary portion 14 of the fixation device 10. In accord with yet another aspect of the invention, the second portion 104 also includes a K-wire guide hole 136 configured to direct a K-wire 138 at a fixed angle into the K-wire alignment hole 142 of the nail portion of the fixation device. Similar to above, prior to drilling any holes into the diaphysis for the screws for the nail portion, a K-wire is drilled through the K-wire guide hole in the second portion and its placement can be viewed under fluoroscopy to ensure its placement relative to the diaphysis is appropriate. If correct, the screw holes can be drilled with assurance that the screws will be properly aligned relative to the axis of the diaphysis. If not correctly aligned as indicated by fluoroscopic visualization, the nail portion can be repositioned, the K-wire re-drilled and re-examined until placement is correct.

The jig is usable in combination with drill guides, bits and gauges as described in previously incorporated U.S. Pat. No. 6,926,720.

There have been described and illustrated herein embodiments of a fixation device and implantation jig therefor. While particular embodiments of the invention have been described, it is not intended that the invention be limited thereto, as it is intended that the invention be as broad in scope as the art will allow and that the specification be read likewise. For example, while pegs have been described for use within the fixation device, other fixed angle bone fixators can be used as well. While the device is preferably sized for implantation at the distal radius, the device may also be sized for placement at the other long bone. It will therefore be appreciated by those skilled in the art that modifications could be made to the provided invention without deviating from its scope as claimed.

1. A fracture fixation device, comprising:
a plate portion having a lower surface, first and second ends, and at least one hole for receiving a bone fixator; and
an intramedullary nail portion which is offset relative to the plate portion, said nail portion extending into a neck portion upon which said lower surface of said plate portion is located, said neck portion meeting said plate portion inward from said first and second ends of said plate portion,
said plate portion having a larger first portion between said neck portion and said first end which extends substantially opposite said nail portion, said first portion including said at least one fixed angle hole, and a second portion extending from said neck portion toward said second end and which defines a lip extending over a portion of said nail portion, said lip and said nail portion defining a space therebetween.

2. (canceled)

3. A fracture fixation device according to claim 1, wherein:
said at least one fixed angle hole is a plurality of fixed angle holes defining at least two axes which extend in discrete directions relative to each other.

4. A fracture fixation device according to claim 1, wherein:
exactly two fixed angle holes extend through said plate portion in said first portion of said plate portion.

5. A fracture fixation device according to claim 4, wherein:
at least one additional hole is provided in said plate portion and also extends at least partially through said neck portion.

6. A fracture fixation device according to claim 1, wherein:
said plate portion has a length longer than a width, and a longitudinal axis defined by said length which extends in generally a same direction as said intramedullary portion.

7. A fracture fixation device according to claim 1, wherein:
said plate portion has a length longer than a width, and a longitudinal axis defined by said length, wherein said longitudinal axis and said intramedullary portion extend within a common plane.

8. A fracture fixation device according to claim 1, wherein:
said plate portion is vertically offset relative to said nail portion.

9. A fracture fixation device according to claim 8, wherein:
said plate portion is horizontally offset relative to said nail portion.

10. A fracture fixation device according to claim 1, wherein:
said plate portion is sized for placement on the metaphysis of the distal radius and the nail portion is sized to be received with the medullary canal of the distal radius.

11. A fracture fixation device for use on a fractured long bone, the bone defining a metaphysis and a diaphysis, and said device for use with at least one bone fixator, said device comprising:
a plate portion for placement on the metaphysis on one side of the fracture, said plate portion having an upper surface and at least one fixed angle hole for receiving one of the bone fixators in a fixed angle orientation; and
an intramedullary nail portion for placement within the diaphysis on the other side of the fracture, said nail portion being offset relative to the plate portion by a neck portion, wherein a portion of said plate portion overhangs said nail portion.

12. A fracture fixation device according to claim 11, wherein:
said neck portion extends from a lower location on said plate portion.

13. A fracture fixation device according to claim 12, wherein:
said plate portion includes a width and said neck portion includes a width which does not exceed said width of said plate portion.

14. (canceled)

15. A fracture fixation device according to claim 11, wherein:
said at least one fixed angle hole is a plurality of fixed angle holes defining at least two axes which extend in discrete directions relative to each other.

16. A fracture fixation device according to claim 11, wherein:
said plate portion has a length longer than a width, and a longitudinal axis defined by said length which extends in generally a same direction as said intramedullary portion.

17. A fracture fixation device according to claim 11, wherein:
said plate portion is vertically offset relative to said nail portion.

18. A fracture fixation device according to claim 17, wherein:
said plate portion is horizontally offset relative to said nail portion.

19. A fracture fixation device according to claim 11, wherein:
at least one of said at least one hole extends through said neck portion.

20. A fracture fixation device according to claim 11, wherein:
said device is sized for implantation at the distal radius.

21. A fracture fixation device for a distal radius having a dorsal surface and a medullary canal, comprising:
a plate portion sized to seat on the dorsal surface, said plate portion having an upper surface and at least one fixed angled hole for receiving a bone fixator in a fixed angle orientation;
a second portion sized to be received within the medullary canal; and
a neck portion by which said plate portion and said second portion are offset relative to each other, wherein said plate portion defines a lip which overhangs a portion of said nail portion.

22. A fracture fixation device according to claim 21, wherein:
said neck portion extends from a lower location of said plate portion.

23. A fracture fixation device according to claim 22, wherein:
said plate portion includes a width and said neck portion includes a width which does not exceed said width of said plate portion.

24. (canceled)

25. A fracture fixation device according to claim 21, wherein:
said plate portion has a length longer than a width, with a longitudinal axis defined by said length extending in a same plane as said second portion.

26. A fracture fixation device according to claim 21, wherein:
said plate portion is vertically offset relative to said second portion.

27. A fracture fixation device according to claim 26, wherein:
said plate portion is horizontally offset relative to said second portion.

28. A fracture fixation device according to claim 1, further comprising:
at least one fixed angle fixator receivable within said at least one fixed angle hole.

29. A fracture fixation device according to claim 11, further comprising:
at least one fixed angle fixator receivable within said at least one fixed angle hole.

30. A fracture fixation device according to claim 21, wherein:
said at least one fixed angle hole is a plurality of fixed angle holes defining at least two axes which extend in discrete directions relative to each other.
